(19) United States
(12) Patent Application Publication
(10) Pub. No.: US 2011/0076771 A1
(43) Pub. Date: Mar. 31, 2011

(54) TISSUE FIBER SCAFFOLD AND METHOD FOR MAKING

(75) Inventors: Peter D. GABRIELE, York, PA (US); Jeffrey H. ROBERTSON, York, PA (US); Jeffrey S. HAGGARD, Cocoa, FL (US)

(73) Assignees: ARMARK AUTHENTICATION TECHNOLOGIES, LLC, York, PA (US); HILLS INC., West Melbourne, FL (US)

(21) Appl. No.: 12/888,472

(22) Filed: Sep. 23, 2010

Related U.S. Application Data
(60) Provisional application No. 61/245,914, filed on Sep. 25, 2009.

(51) Int. Cl. C12N 5/00; B29C 47/00; B29C 47/08

(52) U.S. Cl. 435/396; 435/398; 435/399; 264/177.1; 425/461

(57) ABSTRACT

The present disclosure relates to a fiber, a method of forming a fiber, a system for forming a fiber, and a method of engineering tissue from a fiber. The fiber includes an engineered geometric feature forming a non-Euclidian geometry.

### FIELD

The present disclosure is directed to scaffold and fibers, systems and methods for making scaffold and fibers, and methods of forming materials or organisms by using scaffold and fibers. Specifically, the present disclosure is directed to scaffold and fibers for growing non-Euclidian materials and organisms.

### BACKGROUND

Natural structures that are strictly Euclidean (i.e., having smooth geometric structural forms integrated into the natural systems) are rare or non-existent. Generally, natural structures are fractal in form thus providing increased surface area for the same volume structure.

Engineering non-Euclidian structures can be inconsistent, lack reproducibility, and/or are otherwise difficult to perform, in part, due to rough, irregular, inconsistent, complex, and/or amorphous features. Non-Euclidian structures can include complex shapes having specific small geometric features that are expensive to produce and have been extremely difficult (or even impossible) to produce on a large scale.

Tissue is one such non-Euclidian structure. Thus, tissue engineering can be extremely expensive. Tissue engineering is the application of engineering disciplines to either maintain existing tissue structures or to enable tissue growth. Tissue is a cellular composite representing multiphase systems. The cellular composite can include cells organized into functional units, an extracellular matrix, and a scaffold. The scaffold can include pores, fibers, or membranes. The scaffold can be periodic (i.e. repeating and/or symmetric), fractal, or stochastic (i.e., irregular and/or amorphous).

What is needed is a scaffold or fiber for forming non-Euclidian materials or organisms.

### SUMMARY

One aspect of the disclosure includes a manufactured fiber. The manufactured fiber includes an engineered geometric feature forming a non-Euclidian geometry.

Another aspect of the disclosure includes a method for forming a fiber. The method includes extruding a fiber including an engineered geometric feature forming a non-Euclidian geometry.

Another aspect of the disclosure includes a system. The system includes a die arranged and disposed for extruding a fiber including an engineered geometric feature forming a non-Euclidian geometry.

Another aspect of the disclosure includes a method of engineering tissue. The method includes providing a fiber comprising an engineered geometric feature forming a non-Euclidian geometry, applying tissue to the fiber, and incubating the tissue.

An advantage of the disclosure includes mimicking of biological structures that are non-Euclidian, thereby providing the ability to reproduce biological structures that are less likely to be rejected by the host.

Another advantage of the disclosure includes forming tissue fibers having a surface area greater than a surface area of similar volume Euclidian fibers.

Other advantages that may be realized through the present disclosure include that the use of a fibers having a longitudinal architecture containing engineered features can enhance interlocking of individual fibers, creating greater collective strength, and that micro-texturing of the fiber surface can be provided for alignment response depending on the depth and width of the features as a consequence of the fractal or other design. Exemplary embodiments also present an ability to integrate biomaterials that contain chemistry consistent with natural cell materials with a physical, morphological fabricated topography that signals its ability to act as a host.

Other advantages will be apparent from the following description of exemplary embodiments of the disclosure.

### BRIEF DESCRIPTION

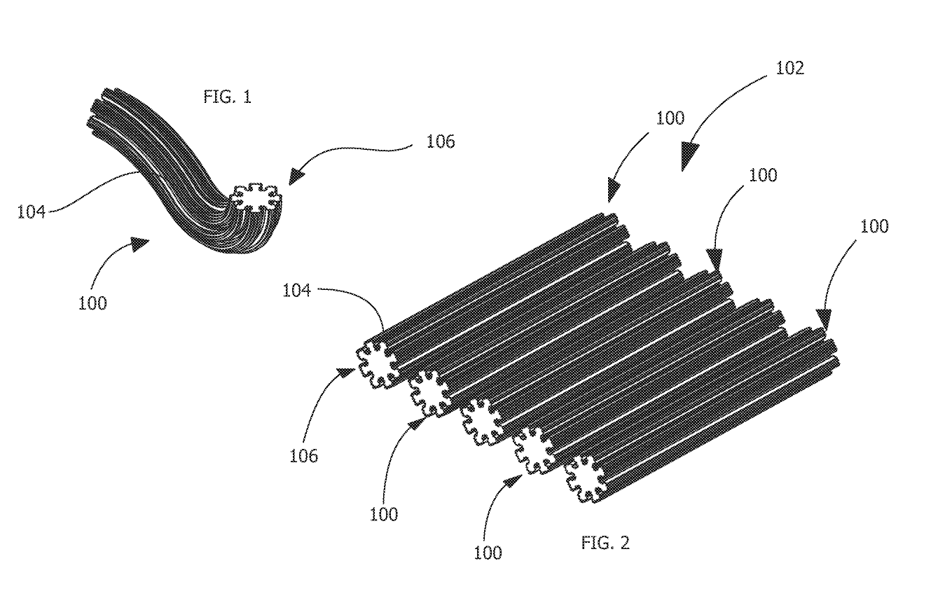

FIG. 1 shows a perspective view of an exemplary fiber.

FIG. 2 shows a perspective view of a plurality of exemplary fibers arranged as an exemplary scaffold.

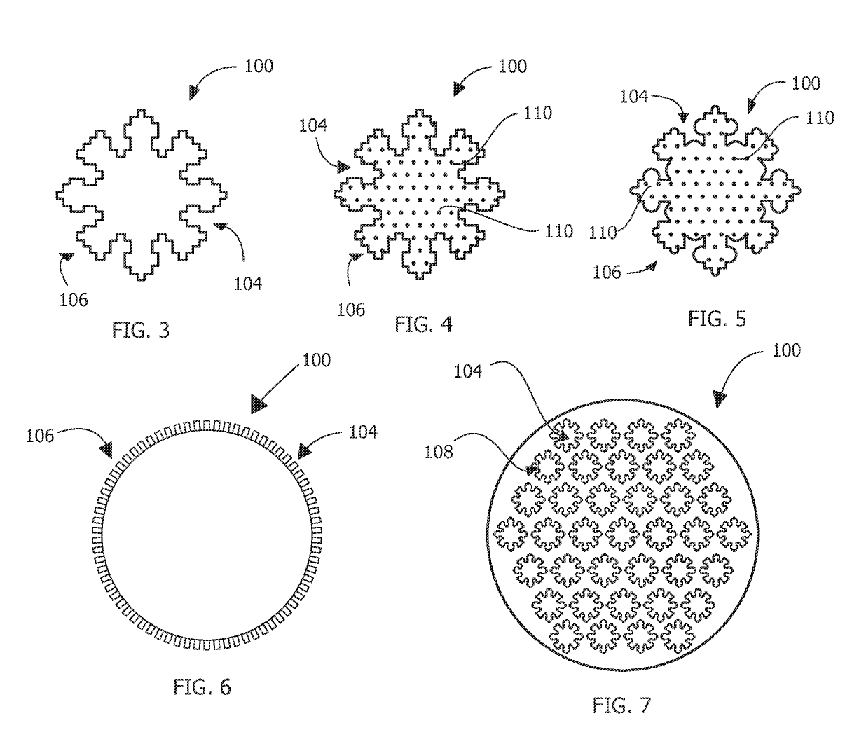

FIGS. 3 through 7 show cross-sectional views of exemplary fibers.

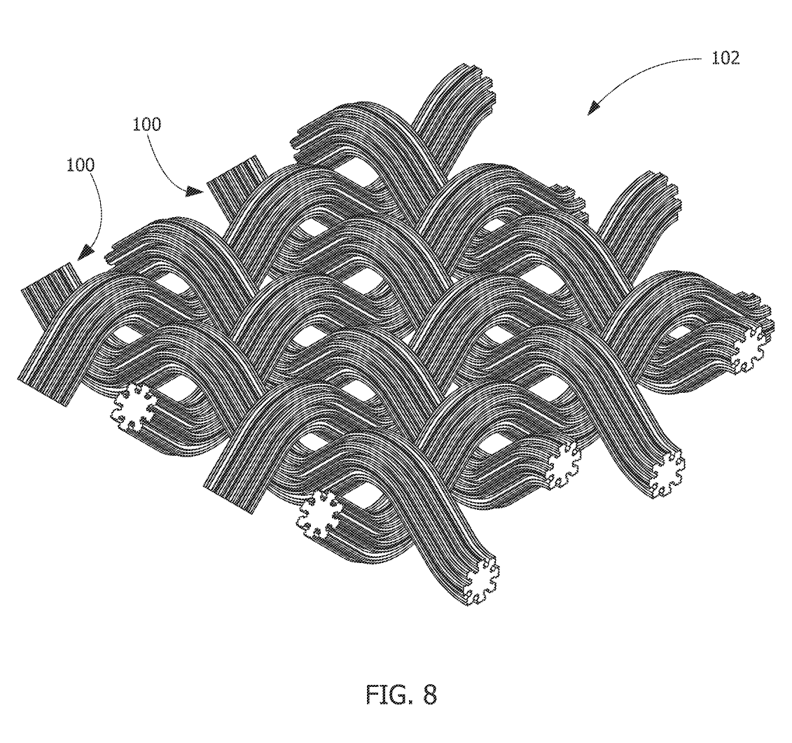

FIG. 8 shows a perspective view of a plurality of exemplary fibers arranged as an exemplary woven scaffold.

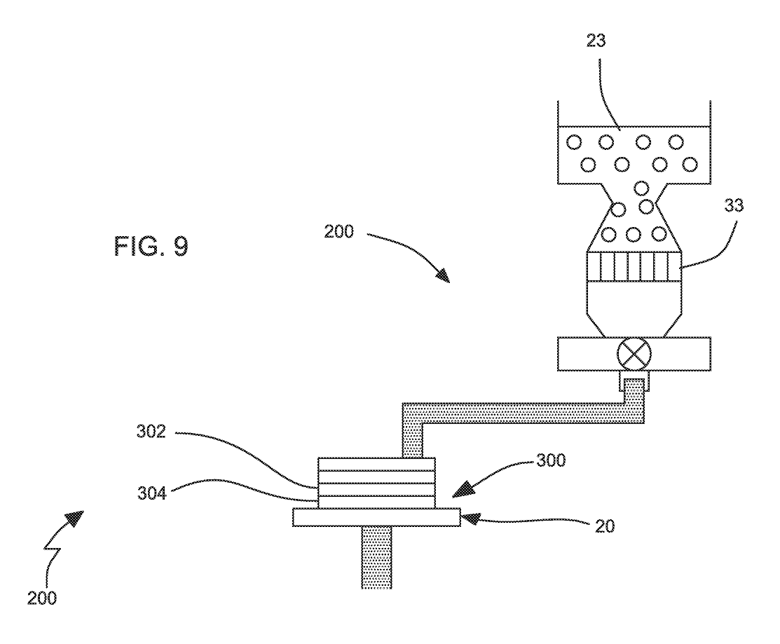

FIG. 9 shows a schematic view of an exemplary microfiber extrusion system.

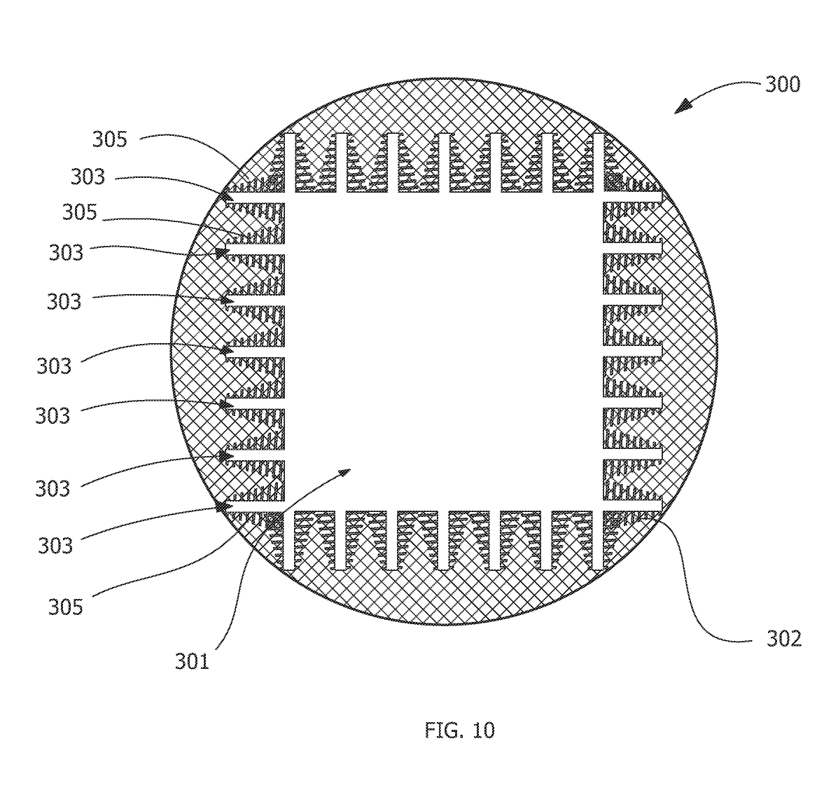

FIGS. 10 through 16 show schematic view of exemplary templates for an exemplary microfiber extrusion system.

Other features and advantages of the present invention will be apparent from the following more detailed description of the preferred embodiment, taken in conjunction with the accompanying drawings which illustrate, by way of example, the principles of the invention.

### DETAILED DESCRIPTION

A scaffold 102 for engineering periodic, fractal and/or stochastic material and/or organisms is disclosed. The scaffold 102 can be formed by the microfiber extrusion system 200 disclosed herein. The scaffold 102 can be used for engineering tissue or other suitable materials or organisms.

Referring generally to FIGS. 1 through 7, the scaffold 102 includes one or more fibers 100. Each fiber 100 contains one or more predetermined geometric features that are engineered, as reflected in the cross-sectional design of the fiber 100, to have a non-Euclidian geometry. The fibers 100 can be arranged with channels 104 (enclosed or exposed), external geometric features 106, and/or internal geometric features 108. The external geometric features 106 and/or internal geometric features 108 can be formed by the arrangement of the channels 104. Additionally or alternatively, the fibers 100 can contain a cross sectional arrangement of several domains 110 (for example, an “islands in the sea” arrangement).

The external geometric features 106 and/or internal geometric features 108 can be nano-sized (i.e., about 1 to 1000 nanometers, typically about 50 to about 500 nanometers and in some embodiments about 50 to about 100 nanometers) or micron-sized (i.e., about 1 to 1000 microns). Thus, the scaffold 102 and/or the fiber 100 can include many design configurations with varying feature sizes. The design configuration can be predetermined to accommodate any suitable growth process (for example, growth of stem cells, nerve cells, tissue, crystal, fungus, bacteria, viruses, etc.). The scaffold 102, the fiber 100, and/or tissue formed may mimic a microstructure favorable for establishing differentiation and resident growth. In one embodiment, the scaffold 102, the fiber 100, and/or tissue formed may include external geometric features 106 and/or internal geometric features 108 having a continuous fractal architecture (or other non-Euclidian forms).

The continuous fractal architecture may mimic microstructural topology of a predetermined structure. Exemplary structures include tissue fractal, neural fractal, bone fractal, tendons, fungus, bacteria, viruses, plants, crystals, other suitable materials and/or organisms, and combinations thereof. The external geometric features 106 and/or internal geometric features 108 may facilitate guided channeling of growth, external troughing of nutrient chemistries, physical unrestricted template support of propagating cells, and/or feed forward orientation for stimulated potentials. Additionally, grooves and ridges and other non-Euclidian features provide for contact guidance and more specifically contact guidance in three dimensions. In contrast to Euclidian surfaces, such features can facilitate tissue growth in the axial direction (or otherwise in opposition to gravity).

As a result, exemplary embodiments provide fibers having a defined structural design for use as a scaffolding material for the promotion of tissue or other growth, the features having defined structural requirements that promote bio-functionalization. The external architecture of such fibers can influence macromolecular organization contributing to a specific biological structure desired to be achieved; the fiber architecture drives organization both in the scaffold structure as well as in the establishment and propagation of cell to tissue organization.

The external architecture of the fibers establishes “contact guidance,” topological control and surface bio-mimetic resemblance. Biological surfaces are rarely flat or smooth and exemplary embodiments can provide a fractal topology and associated topography, which can lead to alignment responses from such cells as neural or vascular progenitor cells.

Referring to FIG. 1, the fiber 100 may be a substantially continuous extrudate having a non-Euclidian external geometry. For example, the fiber 100 may include a periodic exterior. The fiber may be flexible and formed of any suitable component for extrusion and is preferably a viscous material for tissue related end-use. Exemplary materials include polylactic acid polymers and co-polymers and other synthetic biodegradable and biocompatible polymeric materials as well as natural biopolymers like hyaluronic acid, alginates, collagen, chitin, chitosan, proteoglycans, glycosaminoglycans, elastin, fibronectin glycoprotein, and combinations thereof. The surface area of the fiber 100 may be substantially higher than a Euclidian structure having the same volume or cross-sectional area, although the particular increase can vary based on the design, which may depend on a number of factors, including the particular use for which the fiber will be employed.

Referring to FIG. 2, a plurality of the fibers 100 is arranged to form a scaffold 102. The scaffold 102 can be any arrangement of one or more fibers 100. Within the scaffold 102, growth of materials or organisms may occur along channels 104 forming the external geometry of the fibers 100. Upon reaching a predetermined level of growth, materials or organism growing on the fibers 100 may extend across the entire scaffold 102 thereby forming a three-dimensional structure of the material or organism. Positioning materials with varying properties along the fibers 100 and/or along predetermined portions of the scaffold 102 may permit control of the growth of the material or organism.

FIG. 3 shows a cross sectional view of an embodiment of the fiber 100. The embodiment shown in FIG. 3 shows a substantially homogenous fiber having non-Euclidian external geometric features 106.

FIGS. 4 and 5 show cross sectional views of embodiments of a fiber 100 having non-Euclidian external geometric features 106 and having domains 110 arranged throughout an otherwise substantially homogenous fiber as shown. The 110 domains may be arranged within the fiber 100 and positioned by the material of the fiber 100. Alternatively, the domains 110 may be arranged within the fiber 100 and defined by a border between the material within the domains 110 and the remaining material of the fiber 100, or across a gradient to moderate the transition.

The domains 110 may include trophic agents or other materials for promoting or controlling growth of a material or organism on the fiber 100. For example, the domains 110 may include a substance that stimulates growth in the presence of an external stimulus such as an exogenously excitable material. The domains 110 may include material that further mimics a biological architecture. The domains may provide additional strength by including a material stronger than the remaining material of the fiber 100.

FIG. 6 shows a cross sectional view of another embodiment of a fiber 100 having non-Euclidian external geometric features 106 spaced about its outer periphery.

FIG. 7 shows a cross-sectional view of an embodiment of the fiber 100 having a plurality of internal geometric features 108 having non-Euclidian internal geometry and a substantially Euclidian external geometry. The channels 104 may be formed by creating the fiber having an islands-in-the-sea structure, with the islands formed of a material such that when the fiber is placed in a suitable solvent, the island material dissolves, leaving the channels 104 behind in the undissolved surrounding sea material. Alternatively, the fiber 100 could be treated so that the solvent dissolves the surround sea material, resulting in a plurality of smaller fibers in which the internal geometric features 108 formed in the channels 104 shown in FIG. 7 are instead external geometric features of each of the individual smaller fibers.

Referring to FIG. 8, the scaffold 102 and/or the fibers 100 can be weaved with additional scaffold 102 and/or fibers 100 to form a larger scaffold or knit. Any suitable knit may be formed including, but not limited to, weft knit, warp knit—tricot, warp knit with lengthened undertaps, and/or warp knit with weft inserted yarns. In one embodiment, scaffold 102 may be formed by a single fiber 100 weaved around itself. The scaffold 102 can form all or a portion of a covering having a medical use. For example, the scaffold 102 can form a bandage, medical clothing, a skin graft, or any suitable medical application for covering or healing biological substances. In one embodiment, the scaffold 102 forms a skin graft and the domains 110 within the fibers 100 include pharmaceuticals capable of being released to reduce or eliminate rejection, to reduce or eliminate pain, and/or to achieve other suitable effects. The scaffolding 102 can be used for skin disorders such as skin cancer, burns, leprosy, and/or for skin replacement.

The fibers 100 can be formed by any suitable melt spinning or extrusion process that can achieve applicable dimensions. One suitable process is a High Definition Micro Extrusion (“HDME”) process, such as is described by WO 2007/134192. Preferably, the fiber spinning involves a high definition micro-extrusion process as described in WO 2007/134192. This process is a modification of fiber melt-flow spin extrusion adapted to produce a plurality of high definition geometric microstructures that are spatially resolved in cross-section. Spatial resolution may be obtained even in fibers having a diameter as low as 20 to 40 microns. According to an exemplary embodiment, the HDME process is a melt-spin fiber process with a pixel-like die used for the formation of highly resolved and reproducible fractal patterns in the fiber 100. The pixel-like nature can permit flexibility to control fiber geometry for a particular use.

Referring to FIG. 9, a HDME system 200 may be used to extrude scaffold 102 and/or fiber 100. The system 200 can include one or more extruders 33, a spinneret 20 containing one or more templates 300 and/or dies 302, 304 to form the fiber 100 and/or the scaffold 102, and may include other suitable processing equipment for use in processing the fiber 100 and/or the scaffold 102. The extruder 33 generally provides a substantially continuous flow of component fluid to the spinneret 20. In embodiments with multiple extruders, the fluids may remain separate prior to being introduced to the spinneret 20. Referring again to FIG. 9, the volume/area, arrangement, and/or amount of the component 23 may be controlled based upon the fluid from the extruder 33, the arrangement and/or manipulation of the spinneret 20, and/or other suitable process controls. For example, the spinneret 20 may include a template 300 for orienting one or more of the components being extruded to form scaffold 102 and/or fiber 100.

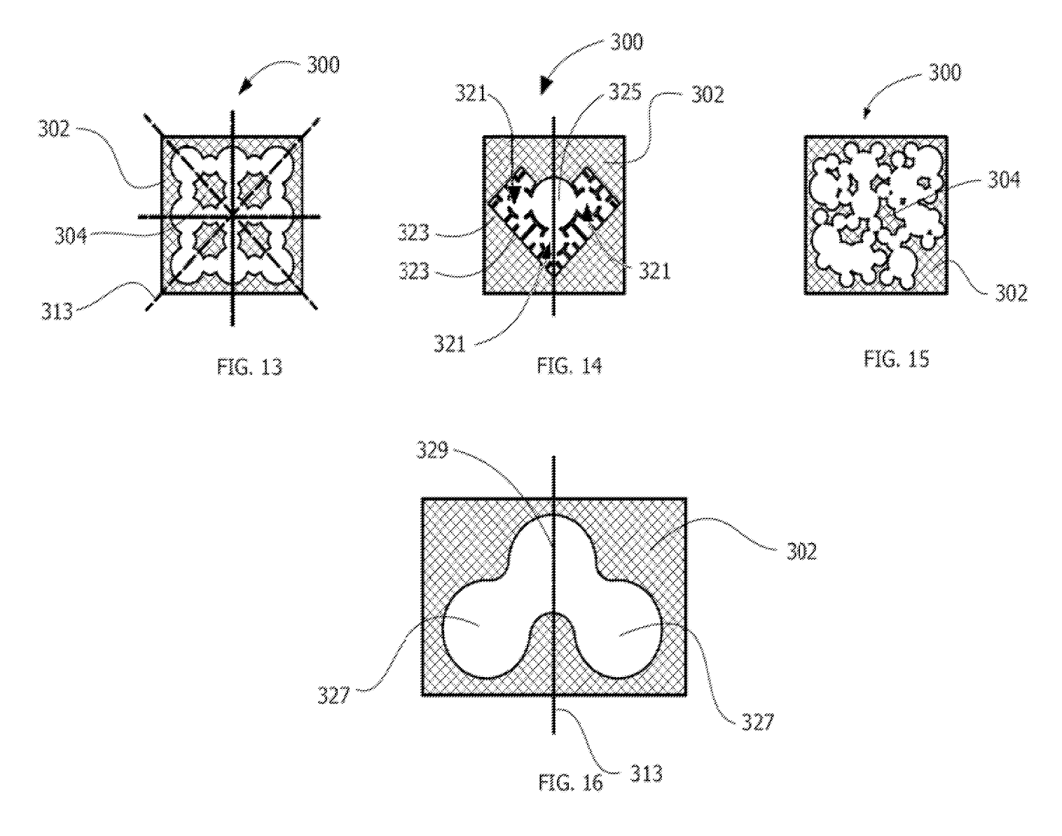

FIGS. 10 through 16 show exemplary templates 300 for spinneret 20. The template 300 includes an external die 302 for forming the external geometric features. Referring to FIGS. 13 and 15, the template 300 may further include an internal die 304 for forming the internal geometric features.

FIG. 10 shows an embodiment of a template 300 with an external die 302 for forming the fiber with external geometric features corresponding to the template 300. The template 300 includes open pixels generally forming a square interior 301. The template 300 further includes open pixels arranged outside the square interior 301 for forming the external geometric features. As illustrated, these external open pixels resemble Christmas trees and include a portion 303 extending from the perimeter and a plurality of smaller portion 305 extending therefrom. Each of the external geometric features is substantially identical and the fiber formed by extruding through the template 300 is symmetric (coaxially) along four lines. The inclusion of the external geometric feature having the portion extending from the perimeter and the plurality of small portions substantially increases the surface area of the fiber extruded through the external die 302. In one embodiment, upon being extruded through the template 300, the material traveling through pixels of the template 300 coalesce to form the fiber.

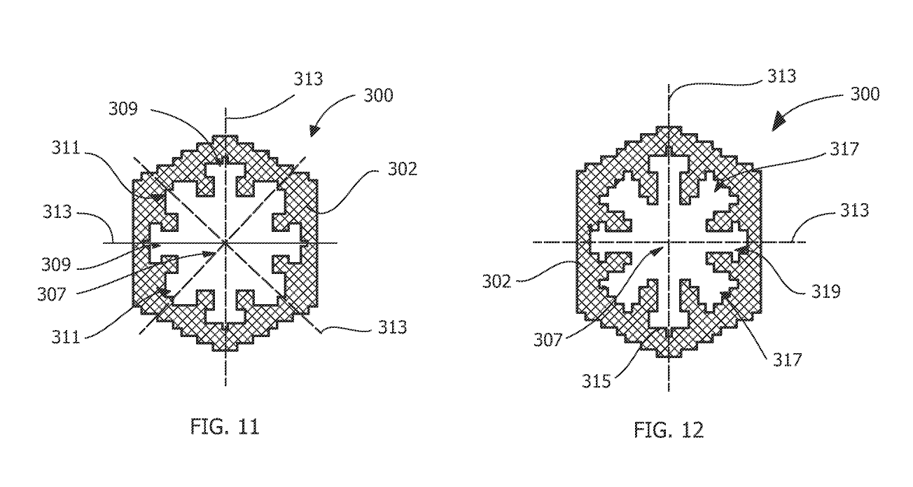

FIG. 11 shows an embodiment of a template 300 with an external die 302 for forming the fiber with external geometric features corresponding to the template 300 extending along the perimeter of a filled interior generally forming a square 307. The external geometric features formed by the external die 302 are arranged to alternate in design with a first design 309 and a second design 911 forming eight lobes. The fiber formed by extruding through the template 300 is symmetric (coaxially) along four lines corresponding to lines 313 shown in FIG. 11.

FIG. 12 shows an embodiment of a template 300 with an external die 302 for forming the fiber with external geometric features extending corresponding to the template 300 along the perimeter of a filled interior generally forming a square 307. The external geometric features formed by the external die 302 are arranged with a first design 315, a second design 317 and a third design 319 forming eight lobes. Specifically, the embodiment shown in FIG. 12 shows four lobes having the second design 317, two lobes having the first design 315, and two lobes having the third design 319. The fiber formed by extruding through the template 300 is symmetric along two lines corresponding to lines 313 shown in FIG. 12.

FIG. 13 shows an embodiment of a template 300 with an external die 302 for forming the fiber with external geometric features corresponding to the template 300 extending along the perimeter of an area generally forming a square. Additionally, the template 300 includes a plurality of internal dies 304 for forming internal geometric features that extend along the interior of the fiber. The external geometric features and the internal geometric features are formed with alternating designs and the fiber formed is symmetric along four lines corresponding to lines 313 shown in FIG. 13. The regions of the fiber defined by the template 300 may be modified or doped with “trophic agents,” i.e. an agent that encourages specific biological activity associated with specific tissue characterization or trophic requirements at a particular region of the fiber's cross-section.

FIG. 14 shows an embodiment of a template 300 with an external die 302 for forming the fiber with external geometric features corresponding to the template 300 extending along the perimeter of the fiber, generally forming three lobes 321 having small square-like structures 323 around them, each lobe being connected to a circle 325. The external geometric features are substantially identical and the fiber formed by extruding through the template 300 is symmetric along one line corresponding to line 313 in FIG. 14.

FIG. 15 shows an embodiment of a template 300 with an external die 302 for forming the fiber with external geometric features corresponding to the template 300 extending along the perimeter of an amorphous structure. Additionally, the template 300 includes a plurality of internal dies 304 for forming internal geometric features that are amorphous. The template 300 forms external geometric features and the internal geometric features that are part of an asymmetric fiber.

FIG. 16 shows an embodiment of a template 300 with an external die 302 for forming the fiber with external geometric features corresponding to the template 300 extending along the perimeter of the fiber generally forming three lobes including two outer lobes 327 connected to each other by a middle lobe 329. The external geometric features are substantially identical and the fiber formed by extruding through the template 300 is symmetric along one line corresponding to line 313 shown in FIG. 16. In other embodiments, additional or alternative designs may be included.

The highly resolved and reproducible nature of the melt-spin extrusion process permits growth of the scaffold 102 and/or the fiber 100, doping of scaffold 102 and/or the fiber 100, and coating of the scaffold 102 and/or the fiber 100 thereby guiding the growth and/or development process. In one embodiment, a base fiber component derived from biopolymer and another material (for example, a water dissolvable polymer) acting as a suitable subtractive polymer (in an islands-in-the-sea arrangement) may form the scaffold 102 and/or the fiber 100. Extrusion processing the biopolymer and suitable subtractive polymer can arrange growth factors or promoting agents within the scaffold 102 and/or fiber 100. Additionally or alternatively, extrusion processing can arrange a plurality of identical or different scaffold 102 and/or fiber(s) 100. The scaffold 102 and/or the fiber(s) 100 may be incubated with tissue for growing the tissue along a predetermined path defined by the scaffold 102 and/or the fiber(s) 100. Additionally, sodium hydroxide may be used to micro-etch the polymer surface. Thus, the fiber may include regions formed of a polymer containing for example, carboxy-functionality, thereby rendering those regions subject to alkaline aqueous dissolution, while at the same time micro-etching the remaining polymer structure with micro-features consistent with promoting cell differentiation as a result of the nano-topography desired to be achieved.

The scaffold 102, the fiber 100, and/or the tissue formed from the scaffold 102 and/or the fiber 100 can be used in an in vivo tissue generation and engineering process. In one embodiment, doing so may include the scaffold 102, the fiber 100, and/or the tissue being formed to receive energetic stimuli to control tissue differentiation or growth. Such tissue differentiation or growth may be enhanced by the arrangement of the scaffold 102 or the fiber 100. For example, channels 104, external geometric features 106, internal geometric features 108, and/or domains 110 may include different properties. The different properties may be based upon the geometry or the contents of the channels 104, external geometric features 106, internal geometric features 108, and/or domains 110. In one embodiment, the depth of grooves and/or channels of internal geometric features 106 and/or external geometric features 108 can control the growth pattern of cells or other biological materials. The fractal fiber architecture described herein provides contact guidance which can provide the environmental cues needed by cells to organize growth into tissue. The templates used to create the fibers introduce engineered features in the fiber architecture that can provide cells with appropriately designed surface features that support the proliferation and differentiation of cell growth.

In a further embodiment, micro-cross-section portions of the scaffold 102, the fiber 100, the tissue, or other suitable particles similarly formed having predetermined aspect ratios can be used as micro-fractal energy reception tissue hyperthermia or ablation particles for cancer therapy and/or for disease management including image diagnostics. In yet another further embodiment, the scaffold 102, the fiber 100, the tissue, or other suitable particles similarly formed can form a fractal antennae. In yet another embodiment, varying levels of exogenously excitable material can permit control of tissue differentiation or growth by permitting certain components of the material to be excited in response to predetermined energetic stimuli.

The extrusion process can incorporate information concerning in situ tissue topology and topography of a known structure to computer generate an arrangement of the scaffold 102 and/or the fiber(s) 100 corresponding to a natural architecture. For example, the scaffold 102 and/or the fiber 100 may be used for growing tissue fractal, neural fractal, and/or bone fractal. In other embodiments, the scaffold 102 and/or the fiber 100 may form tendons, fungus, bacteria, viruses, plants, crystals, or other suitable materials and/or organisms based upon computer generated images associated with the structures. The fibers 100 and/or scaffold 102 may be performed by translating image and other information regarding cells and tissue for which grown is to be fostered into computer-aided-design (CAD) drawings, engineering designs or other suitable design systems. It will be appreciated that the scaffold 102 and/or the fiber 100 formed are not limited to biological materials or bio-medical applications.

Although certain features are described in the context of certain embodiments, it will be appreciated that the various features and aspects are equally applicable with respect to other embodiments and that the teachings may be combined in any manner desired to achieve the fibers described herein.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. For example, ranges, relationships, quantities, and comparisons between aspects of the disclosure (including the Figures) are included within the scope of the invention. Therefore, it is intended that the invention not be limited to the particular embodiment disclosed as the best mode contemplated for carrying out this invention, but that the invention will include all embodiments falling within the scope of the appended claims.

What is claimed is:

1. A manufactured fiber comprising an engineered geometric feature forming a non-Euclidian geometry.

2. The fiber of claim 1, wherein the non-Euclidian geometry is fractal.

3. The fiber of claim 1, wherein the non-Euclidian geometry is amorphous.

4. The fiber of claim 1, wherein the engineered geometric feature is formed on an external surface of the fiber.

5. The fiber of claim 1, wherein the non-Euclidian geometry is periodic.

6. The fiber of claim 5, wherein the non-Euclidian geometry is symmetric along four lines.

7. The fiber of claim 5, wherein the non-Euclidian geometry is symmetric along two lines.

8. The fiber of claim 5, wherein the non-Euclidian geometry is symmetric along one line.

9. The fiber of claim 1, wherein the engineered geometric feature comprises a material selected from the group consisting of polylactic acid polymers and co-polymers, synthetic biodegradable and biocompatible polymeric materials, hyaluronic acid, alginates, collagen, chitin, chitosan, proteoglycans, glycosaminoglycans, elastin, fibronectin glycoprotein, and combinations thereof.

10. The fiber of claim 1, further comprising one or more domains that include a material for promoting or controlling growth.

11. The fiber of claim 1, further comprising one or more domains that include a material that stimulates growth in the presence of an external stimulus.

12. The fiber of claim 1, wherein the engineered geometric feature mimics a feature of a biological architecture.

13. The fiber of claim 1, wherein the fiber is arranged to form a scaffold.

14. The fiber of claim 13, wherein the scaffold is woven.

15. The fiber of claim 1, wherein the engineered geometric feature is formed on an internal surface of the fiber.

16. The fiber of claim 1, wherein the engineered geometric feature is between about 50 to 500 nanometers.

17. A method for forming a fiber, comprising:
extruding a fiber comprising an engineered geometric feature forming a non-Euclidian geometry.

18. The method of claim 17, wherein the engineered geometric feature is formed by translating information from computer-aided-design drawings.

19. A system, comprising a die, the die being arranged and disposed for extruding a fiber comprising an engineered geometric feature forming a non-Euclidian geometry.

20. A method of engineering tissue, comprising:
providing a fiber comprising an engineered geometric feature forming a non-Euclidian geometry;
applying tissue to the fiber; and
incubating the tissue.
